(19) United States
(12) Patent Application Publication
(10) Pub. No.: US 2016/0038743 A1
(43) Pub. Date: Feb. 11, 2016

(54) IMPLANTABLE MEDICAL DEVICE COATING FOR WETTING AND MICROBIAL RESISTANCE

(71) Applicant: Cardiac Pacemakers, Inc., St. Paul, MN (US)

(72) Inventors: Arthur J. Foster, Blaine, MN (US); Mathew L. Gilk, Brooklyn Park, MN (US); Keith R. Maile, New Brighton, MN (US); Joseph T. Delaney, JR., Minneapolis, MN (US); Michael Eppihimer, Franklin, MA (US); Gary T. Seim, Minneapolis, MN (US)

(21) Appl. No.: 14/822,779

(22) Filed: Aug. 10, 2015

Related U.S. Application Data
(60) Provisional application No. 62/035,557, filed on Aug. 11, 2014.

(51) Int. Cl. A61N 1/375; C09D 171/02

(52) U.S. Cl. CPC A61N 1/375; C09D 171/02

(57) ABSTRACT

The present invention relates generally to the materials and methods for improving the wettability of an implantable medical device. More specifically, the invention relates to devices and methods for coating at least a portion of a medical device to improve wettability and reduce electrical impedance. Given the challenges of maintaining consistent and predictable electrical characteristics as implantable medical devices become smaller in size, there remains a continual need to improve wettability in an effort to improve medical outcomes.

### CROSS-REFERENCE TO RELATED APPLICATIONS

This application claims the benefit under 35 U.S.C. section 119(e) to U.S. provisional application No. 62/035,557, filed on Aug. 11, 2014, which is herein incorporated by reference in its entirety.

### TECHNICAL FIELD

The present invention relates generally to an implantable medical device. More specifically, the present invention relates to an implantable medical device having improved wettability and reduced electrical impedance and methods of making.

### BACKGROUND

Implantable medical devices can be used to treat a wide variety of medical conditions through many different mechanisms, including pharmaceutical administration, pain management, and electrical stimulation. Stimulation systems in particular have been developed to provide therapy by modulating a subject's electrical impulses. For example, a cardiac pacemaker is commonly implanted in a subject to treat bradycardia (i.e., abnormally slow heart rate), while a cardioverter defibrillator (ICD) is used to treat tachycardia (i.e., abnormally rapid heart rate). Implantable medical devices that deliver electrical stimulation can also provide therapy by stimulating nerves or muscles. For example, stimulating nerve fibers proximal to the pudendal nerves of the pelvic floor can treat urinary urge incontinence, and stimulating the cavernous nerve(s) can be used to treat erectile and other sexual dysfunctions. Additionally, nerve stimulation can help reduce pressure sores and venous stasis, as well as stimulate the spinal cord to relieve intractable chronic pain.

Typically, devices that provide electrical stimulation include a pulse generator, an active element (e.g., a lead or feedthrough), and some form of electrical connection between these components. Pulse generators typically include housing for a battery and electrical circuitry, as well as a header for connecting the active element or elements to the pulse generator. Once implanted, it is often preferable that the stimulation system be controlled and/or that the electrical source can be charged without removing the stimulation system from the implantation environment.

One of the challenges pertaining to implantable medical devices that provide electrical stimulation is establishing and maintaining consistent and predictable electrical characteristics just after implantation (i.e., the acute phase), as well as over a long period of time, such as a period of weeks, months or years (i.e., chronic phase). For example, the electrical impedance exhibited by an implantable medical device providing electrical stimulation often changes during the period immediately after implantation compared to several weeks after implantation. Given that electrical impedance can be used as a performance metric for medical devices (e.g., to assess lead integrity), the ability to reduce inconsistencies and variability related to electrical impedance will lead to better medical outcomes for the subject. Additionally, establishing and maintaining more consistent and predictable electrical characteristics is becoming more and more challenging as implantable medical devices become smaller and smaller.

Given the challenges of maintaining consistent and predictable electrical characteristics as implantable medical devices become smaller in size, there remains a continual need to improve wettability, as doing so will have beneficial effects on medical outcomes.

### SUMMARY

Disclosed herein are various embodiments of a coated medical device, as well as methods for coating medical devices.

In Example 1, an implantable medical device includes a pulse generating portion enclosed within a housing, an active element electrically coupled to the pulse generating portion, and a coating disposed over the external surface of at least a portion of the implantable medical device. The coating includes a wetting agent, wherein electrical impedance of the implantable medical device is reduced compared to the medical device without the coating.

In Example 2, the implantable medical device according to the Example 1, wherein the coating is disposed over at least a portion of at least one of the housing of the pulse generating portion and the active element.

In Example 3, the implantable medical device according to Example 1 or Example 2, wherein the acutely observed electrical impedance is reduced by at least about 5% compared to the medical device without the coating.

In Example 4, the implantable medical device according to any of Examples 1-3, wherein the variability in electrical impedance acutely observed between the acute phase and the chronic phase is reduced compared to the medical device without the coating.

In Example 5, the implantable medical device according to any of Examples 1-4, wherein the wetting agent comprises a member selected from the group consisting of polyethylene glycol (PEG), PEG copolymers, and combinations thereof.

In Example 6, the implantable medical device according to any of Examples 1-5, wherein the wetting agent is PEG having an average molecular weight (Mn) between 500 and 20,000.

In Example 7, the implantable medical device according to any of Examples 1-6, wherein the coating comprises PEG in an amount of 1 milligram (mg) or less.

In Example 8, the implantable medical device according to any of Examples 1-7, wherein the wetting agent is a cross-linkable hydrophilic polymer.

In Example 9, the implantable medical device according to any of Examples 1-8, wherein the coating reduces interfacial surface tension on the exterior surface of at least a portion of the implantable medical device.

In Example 10, the implantable medical device according to any of Examples 1-9, wherein the coating reduces bacterial adhesion compared to the medical device without the coating.

In Example 11, the implantable medical device according to any of Examples 1-10, and further comprising a chamber covering the housing of the pulse generating portion, wherein the coating is disposed on the external surface of the chamber.

In Example 12, a method of manufacturing an implantable medical device includes applying a coating solution that includes a wetting agent to an external surface of the medical device, and drying the applied coating, wherein the acutely observed electrical impedance of the medical device is reduced compared to the medical device without the coating.

In Example 13, the method according to Example 12, wherein applying the coating comprises brushing, dipping, spraying, or combinations thereof.

In Example 14, the method according to any of Example 12 or Example 13, wherein the acutely observed electrical impedance is reduced by at least about 5% compared to the medical device without the coating.

In Example 15, the method according to any of Examples 12-14, wherein the wetting agent is PEG having an average molecular weight (Mn) from about 500 to about 20,000.

In Example 16, an implantable medical device includes a pulse generating portion enclosed within a housing, an active element electrically coupled to the pulse generating portion, and a coating that includes a wetting agent. The coating can be disposed over at least a portion of at least one of the housing of the pulse generating portion and the active element. The wetting agent can include PEG having an average molecular weight (Mn) from about 500 to about 20,000, wherein the PEG is present in an amount of about 1 mg or less.

In Example 17, the implantable medical device according to Example 16, wherein the PEG includes a cross-linkable hydrophilic polymer.

In Example 18, the implantable medical device according to Example 16 or Example 17, wherein the acutely observed electrical impedance is reduced by at least about 5% compared to the medical device without the coating.

In Example 19, the implantable medical device according to Examples 16-18, wherein the variability in electrical impedance between the acute phase and the chronic phase is reduced compared to the medical device without the coating.

In Example 20, an implantable medical device includes a pulse generating portion enclosed within a housing, an active element, wherein the pulse generating portion is electrically coupled to the active element, and a coating comprising a wetting agent. The coating can be disposed over at least a portion of the medical device, wherein the acutely observed electrical impedance of the implantable medical device is reduced compared to the medical device without the coating.

In Example 21, the implantable medical device according to Example 20, wherein the coating is disposed over at least a portion of at least one of the housing of the pulse generating portion and the active element.

In Example 22, the implantable medical device according to Example 20 or Example 21, wherein the acutely observed electrical impedance is reduced by at least about 5% compared to the medical device without the coating.

In Example 23, the implantable medical device according to Examples 20-22, wherein the variability in the acutely observed electrical impedance between the acute phase and the chronic phase is reduced compared to the medical device without the coating.

In Example 24, the implantable medical device according to Examples 20-23, wherein the wetting agent includes a member selected from the group consisting of polyethylene glycol (PEG), PEG copolymers and combinations thereof.

In Example 25, the implantable medical device according to Examples 20-24, wherein the wetting agent is PEG having an average molecular weight (Mn) between about 500 and about 20,000.

In Example 26, the implantable medical device according to Example 25, wherein the PEG applied is present in a total amount of about 1 mg or less.

In Example 27, the implantable medical device according to Examples 20-26, wherein the wetting agent is a cross-linkable hydrophilic polymer.

In Example 28, the implantable medical device according to Examples 20-27, wherein the coating reduces interfacial surface tension between an aqueous fluid and the external surface of at least a portion of the implantable medical device.

In Example 29, the implantable medical device according to Examples 20-28, wherein the coating reduces bacterial adhesion compared to a medical device without the coating.

In Example 30, the implantable medical device according to Examples 20-29, further including a chamber covering the pulse generation portion and the housing of the pulse generating portion, wherein the coating is applied to the exterior surface of the chamber instead of the housing.

In Example 31, the implantable medical device according to Examples 20-30, wherein the active element is a lead.

In Example 32, a method of manufacturing an implantable medical device includes applying a coating solution that includes a wetting agent to an external surface of the medical device, and drying the applied coating, wherein the acutely observed electrical impedance of the medical device is reduced compared to the medical device without the coating.

In Example 33, the method according to Example 32, wherein applying the coating includes brushing, dipping, spraying, or combinations thereof.

In Example 34, the method according to Example 32 or Example 33, wherein the acutely observed electrical impedance is reduced by at least about 5% compared to the medical device without the coating.

In Example 35, the method according to Examples 32-34, wherein the wetting agent is PEG having an average molecular weight (Mn) from about 500 to about 20,000.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a perspective view of an implantable medical device, according to one embodiment of the present invention.

FIG. 2A is an exemplary illustration of a coating applied to an implantable medical device, according to one embodiment of the present invention.

FIG. 2B is an exemplary illustration of a coating applied to a chamber that encloses an implantable medical device, according to one embodiment of the present invention.

FIG. 3A is an exemplary illustration of a hydrophobic surface having reduced wettability, according to one embodiment of the present invention.

FIG. 3B is an exemplary illustration of a hydrophilic surface having enhanced wettability, according to one embodiment of the present invention.

FIG. 3C is an exemplary illustration of a hydrophobic surface coated with a wetting agent to enhance wettability, according to one embodiment of the present invention.

FIG. 4 is a perspective view of an implantable medical device, according to one embodiment of the present invention.

FIG. 5 parts A and B are a graphical representations illustrating reduced electrical impedance (FIG. 5 part A) and reduced power (FIG. 5 part B) in an implantable medical device coated with a wetting agent, according to one embodiment of the present invention.

FIG. 6 is a graphical representation illustrating reduced bacterial adhesion to a portion of an implantable medical device coated with a wetting agent as compared to an untreated sample, according to one embodiment of the present invention.

FIGS. 7A and 7B are exemplary images of TSA plates stamped with a portion of an implantable medical device that was uncoated (FIG. 7A) or coated (FIG. 7B).

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

The present invention relates generally to an implantable medical device. More specifically, the invention relates to the materials and methods for an implantable medical device having improved wettability and reduced electrical impedance.

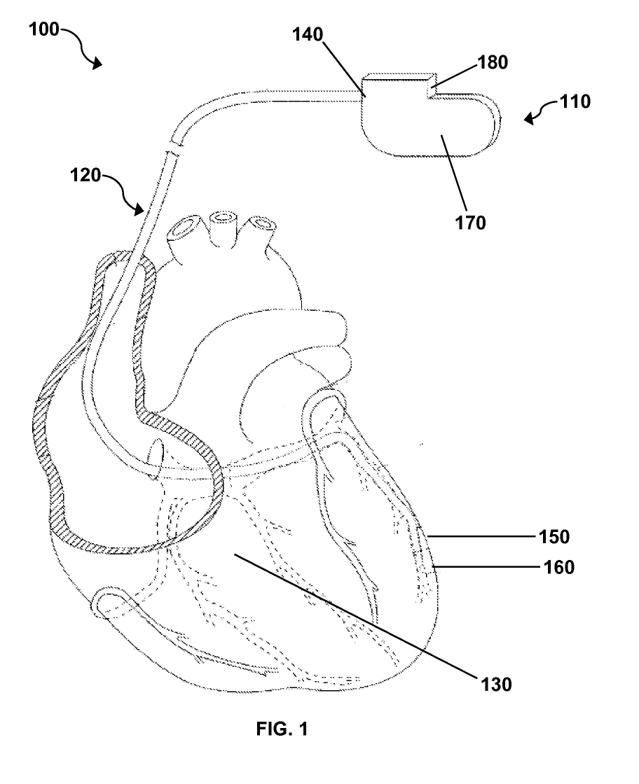

In some embodiments, the implantable medical device of the present invention comprises an electrical stimulation system that modulates the electrical impulses in a subject. In some embodiments, the implantable medical device can be a cardiac pacemaker, as illustrated in FIG. 1. For example, a cardiac pacemaker 100 can include a pulse generating portion 110 and an active element 120. In some cases, the active element can be a cardiac lead, and in other cases, the active element can be a wireless electrode assembly, or a “seed.” For example, the pulse generating portion 110 can be electrically coupled to a lead 120, which conveys electrical signals between the heart 130 and the pulse generating portion 110. A proximal region 140 of the lead 120 can be coupled to the pulse generating portion 110 and a distal region 150 can be coupled to the heart 130. The distal region 150 can be guided through the superior vena cava, the right atrium, the coronary sinus ostium and the coronary sinus, and into the branch vessel of the coronary sinus, with the distal region 150 positioned within the branch vessel. In some cases, the distal region 150 can terminate in an electrode 160. The illustrated position of the lead 120 may be used, for example, for sensing physiologic parameters and delivering a pacing and/or defibrillation stimulus to the left side of the heart 130. The lead 120 may also be partially deployed in other cardiac vessels such as the great cardiac vein or other branch vessels for providing therapy to the left side (or other portions) of heart the 130.

In some embodiments, the cardiac pacemaker 100 can comprise a plurality of active elements or leads 120. For example, the cardiac pacemaker 100 can include a first lead adapted to convey electrical signals between a pulse generating portion 110 and the left ventricle and a second lead adapted to convey electrical signals between the pulse generating portion 110 and the right ventricle. The active elements or leads 120 can also be implanted in any other portion of the heart 130 as known in the art. For example, the leads 120 may be implanted in the right atrium, the right ventricle, the pulmonary artery, the left ventricle, or in the coronary veins. In some embodiments, the cardiac pacemaker 100 can comprise multiple electrodes 160 disposed to sense electrical activity and/or deliver therapy to both the left and right sides of the heart 130. For example, a lead 120 can be an epicardial lead where the electrode 160 penetrates the epicardium.

In some embodiments, the active element of the cardiac pacemaker 100 can comprise a plurality of wireless electrode assemblies, or “seeds,” that are implanted within the chambers of the heart. For example, various numbers of seeds can be implanted in the atria and ventricles of the heart. Each seed can have an internal coil that is inductively coupled with an external power source coil to charge an electrical storage device contained in the seed, which also has a triggering mechanism to deliver stored electrical charge to the adjacent heart tissue. The seed assembly system can also comprise circuitry to sense and analyze the heart's electrical activity, and to determine if and when a pacing electrical pulse needs to be delivered and from which seed.

In some embodiments, the cardiac pacemaker 100 can include a housing 170 which encloses the pulse generating portion 110. The housing 170 can be a body that provides a sealed chamber for the electrical components (e.g., microprocessor chip, pulse output circuits, sense amplifiers, telemetry coils, etc.) necessary for sending a signal to the active element, as well as a power source (e.g., a battery). In some embodiments, housing 170 can have a uniform or substantial uniform thickness. The pulse generating portion 110 can be implanted subcutaneously at an implantation location in the subject's chest or abdomen. The housing 170 can be secured to the tissues of a subject during implantation to prevent migration of the pulse generating portion 110. The housing 170 can also encase a header portion 180, which generally provides an interface for connecting the lead 120 to the pulse generating portion 110 of the medical device.

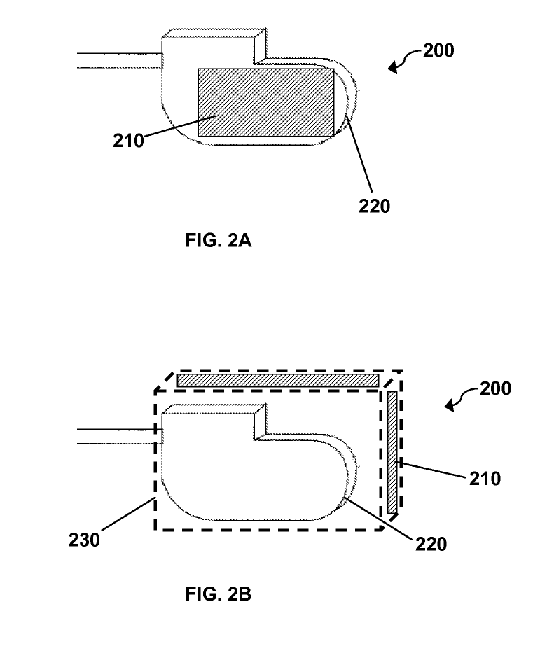

In some embodiments, an implantable medical device such as a cardiac pacemaker can further comprise a coating, as shown in FIGS. 2A and 2B. In some embodiments, the coating 210 can be disposed over or applied to at least a portion of an exterior surface of the pulse generating portion 200. In some embodiments coating 210 is the outer most layer such that the coating 210 will be in contact with bodily fluids or tissues of the surrounding environment (i.e., the subject). For example, the coating 210 can be applied to a portion of the exterior surface area of the housing 220 of pulse generating portion 200. In some cases, the coating 210 can be disposed over the entire surface area of the housing 220 of the pulse generating portion 200. Alternatively, the coating 210 can be disposed over a portion of the surface area of the housing 220. In some embodiments, the coating 210 can be disposed over an area corresponding to between about 50% and about 100% of the surface area of the housing 220 of the pulse generating portion 200. In other cases, the coating 210 can be disposed over an area corresponding to between about 0% and about 50% of the surface area of the housing 220 of the pulse generating portion 200. In other cases, the coating 210 can be disposed over an area corresponding to between about 25% and about 75% of the surface area of the housing 220 of the pulse generating portion 200.

In some aspects, an implantable medical device such as a cardiac pacemaker can include a coating 210 that is disposed over or applied to a chamber 230, instead of coating the medical device directly. As shown in FIG. 2B, the chamber 230 can cover a portion of the implantable medical device, such as the pulse generating portion 200. The chamber 230 may be formed of a biocompatible material that facilitates its placement over the housing 220 of the pulse generating portion 200, such as a flexible silicone boot. In some embodiments, the chamber 230 including the coating 210 can be placed over the housing 220 at the site of manufacture, which may obviate the need to directly coat the medical device prior to implantation. Alternatively, the coating 210 can be placed over the housing 220 after manufacture but prior to implantation.

In some embodiments, the chamber 230 can provide a hermetically sealed environment for the pulse generating portion 200 of the implantable medical device. The coating 210 can then be applied to the entire exterior surface area of the chamber 230 or a portion of the exterior surface area of the chamber 230. Additionally, the chamber 230 can be separate from the housing 220, such that it covers at least a portion of the housing 220. In some embodiments, the chamber 230 can be replaced with a different chamber, for example when necessary (i.e., standard repair and maintenance). In some embodiments, the coating 210 can be disposed over an area corresponding to between about 50% and about 100% of the surface area of a portion of the chamber 230. In other embodiments, the coating 210 can be disposed over an area corresponding to between about 0% and about 50% of the surface area of the chamber 230. In other embodiments, the coating 210 can be disposed over an area corresponding to between about 25% and about 75% of the surface area of the chamber 230. In some embodiments, the chamber 230 can include a biocompatible material.

The coating 210 can be applied for a variety of purposes. For example, implantable medical devices comprising stimulation systems can exhibit changes in electrical parameters, such as impedance, when examined over time, from the initial period after implantation (i.e., acute phase) to an extended period after implantation (i.e., chronic phase). During the acute phase, which is generally considered to last up to about two months after implantation, portions of the implantable medical device are subject to on-going tissue encapsulation. During this phase, changes in tissues surrounding the pulse generating portion and/or the active element can greatly affect electrical impedance. For example, the surrounding tissue may increase electrical impedance or create undesired variability. The chronic phase may be considered as the time two months from implantation. During the chronic phase, impedance and other electrical characteristics tend to stabilize. In some cases, reducing the variability of the electrical parameters (e.g., impedance) exhibited by an implantable medical device between the acute and chronic phases after implantation can lead to greater predictability and better medical outcomes for the subject.

In some embodiments, the coating 210 can include a wetting agent that can, for example, enhance wettability. Generally, a wetting agent is a substance that lowers the surface tension of a liquid and thus allows the liquid to spread more easily over a given surface. In some cases, the application of a wetting agent over a surface can increase the hydrophilicity of that surface. The hydrophilicity of a given surface can be measured using, for example, a drop test, in which a drop of liquid having a defined volume is applied to a surface (coated or uncoated) and various quantitative measurements are taken. For example, the rate at which the drop of water to spreads can be determined, as well as the contact angle (i.e., the angle, conventionally measured through the liquid, where a liquid interface meets a solid surface). Additionally or alternatively, the coating 210 may reduce the variability in electrical impedance after implantation of an implantable medical device. Wettability generally refers to the preference of a solid to be in contact with one fluid rather than another. For example, wettability can be increased or enhanced by reducing the interfacial surface tension (i.e., by lowering the contact angle) between the surface of a medical device and the fluids in the implantation environment. This allows the fluid to spread more evenly over the surface, ultimately resulting in a reduction in electrical impedance. Another beneficial aspect of enhancing wettability between a surface of a medical device and the fluids in the implantation environment includes reducing the ability of harmful bacteria to adhere to components of the medical device and cause infection. In some embodiments, the coating 210 including a wetting agent can, for example, reduce the ability of harmful bacteria to adhere to the medical device after implantation, thus providing anti-microbial properties. In other embodiments, enhancing wettability can reduce the ability of macrophages and other immune-responsive cells from adhering to the medical device and activating a subject's inflammatory response.

Enhancing wettability on the surface of an implantable device through the application of a coating 210 which includes a wetting agent also allows fluids to spread more evenly over the surface of the implantable medical device, ultimately resulting in a reduction in electrical impedance of electrical impedance variability. In some embodiments, reductions in electrical impedance after implantation of a medical device comprising a coating 210 having a wetting agent can be from about 5% to about 50%. In some embodiments, reductions in electrical impedance after implantation of a medical device comprising a coating 210 including a wetting agent can be from about 5% to about 40%. In some embodiments, reductions in electrical impedance after implantation of a medical device comprising a coating 210 including a wetting agent can be from about 5% to about 30%. In some embodiments, reductions in electrical impedance after implantation of a medical device comprising a coating 210 having a wetting agent can be from about 5% to about 25%. In some embodiments, reductions in electrical impedance after implantation of a medical device comprising a coating 210 including a wetting agent can be from about 5% to about 20%. In some embodiments, reductions in electrical impedance after implantation of a medical device comprising a coating 210 including a wetting agent can be from about 5% to about 15%. In some embodiments, reductions in electrical impedance after implantation of a medical device comprising a coating 210 including a wetting agent can be from about 5% to about 10%. In some embodiments, reductions in electrical impedance after implantation of a medical device comprising a coating 210 including a wetting agent can be from about 10% to about 50%. In some embodiments, reductions in electrical impedance after implantation of a medical device comprising a coating 210 including a wetting agent can be from about 10% to about 40%. In some embodiments, reductions in electrical impedance after implantation of a medical device comprising a coating 210 including a wetting agent can be from about 10% to about 30%. In some embodiments, reductions in electrical impedance after implantation of a medical device comprising a coating 210 including a wetting agent can be from about 10% to about 20%. In some embodiments, reductions in electrical impedance after implantation of a medical device comprising a coating 210 including a wetting agent can be from about 20% to about 50%. In some embodiments, reductions in electrical impedance after implantation of a medical device comprising a coating 210 including a wetting agent can be from about 30% to about 40%.

In some embodiments, the reduction in electrical impedance after implantation of a medical device including a coating 210 with a wetting agent can be acutely observed. In some embodiments, the reduction may be acutely observed through the application of a stimulation pulse to the patient's heart, which allows measurement of impedance in a medical device or a portion of the medical device by measuring the current flowing through the medical device when the voltage pulse of known magnitude is applied. The impedance data may be measured and stored at predetermined time intervals. In some embodiments, impedance or stimulation threshold at the time of implantation can be measured to optimize the location and longevity of the medical device. These acute measurements can be made with an oscilloscope or with a pacing system analyzer or prostate specific antigen (PSA).

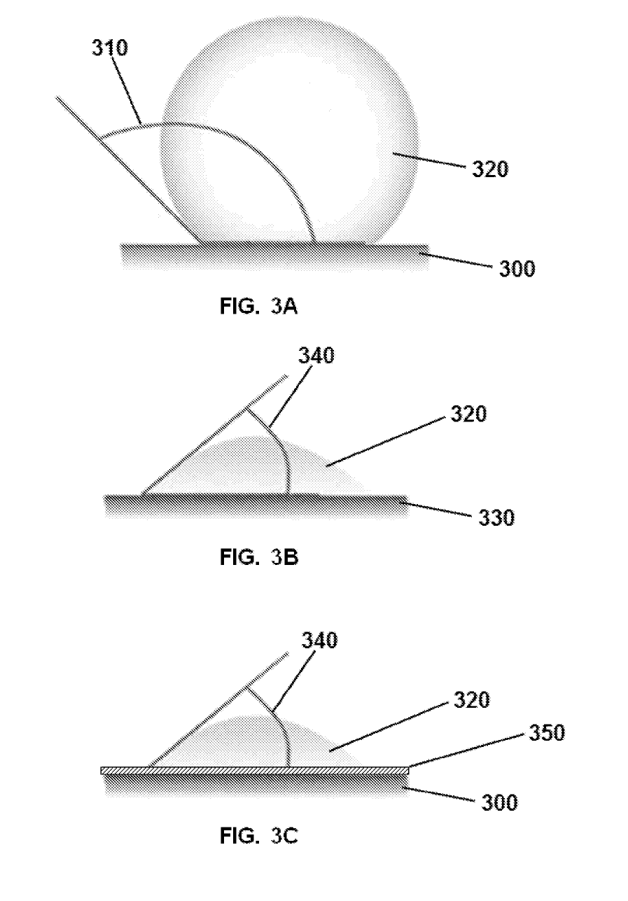

FIGS. 3A-3C provide exemplary illustrations of a surface having enhanced or improved wettability. As shown in FIG. 3A, a surface 300 having low or reduced wettability results in a large contact angle 310 between the surface 300 and a fluid droplet 320. In this case, surface the 300 can be considered hydrophobic. As shown in FIG. 3B, the surface 330 having high or increased wettability results in a small contact angle 340 between the surface 330 and the fluid droplet 320. In this case, the surface 330 can be considered hydrophilic. FIG. 3A illustrates a fluid droplet 320 on a first exemplary surface 300. The contact angle 310 is formed between the surface 300 and the fluid droplet 320. As known in the art, the contact angle formed between the surface 300 and fluid droplet 320 can be measured as shown. FIG. 3B illustrates the fluid droplet 320 on a second exemplary surface 330, where contact angle 340 is formed between the surface 330 and the fluid droplet 320. Comparing the contact angle 310 of FIG. 3A to the contact angle 340 of FIG. 3B, the contact angle 310 is larger than the contact angle 340. Thus, the surface 330 has a higher or increased wettability as compared to the surface 300.

As shown in FIG. 3C, a surface 300 having a coating 350 may have a lower or reduced wettability as compared to surface 300 alone, resulting in small contact angle 340 between surface 300 with coating 350 and fluid droplet 320. In some embodiments, the coating 350 can enhance the wettability of the surface 300 by increasing its hydrophilicity. In some embodiments, for example, the surface 300 can be the exterior surface of a medical device, such as the exterior surface of a housing that encloses a pulse generating portion of an implantable medical device. The coating 350 can, for example, enhance wettability and reduce the variability in electrical impedance after implantation of an implantable medical device (e.g., a cardiac pacemaker).

A wetting agent can be selected from the group consisting of polyethers, non-ionic aprotic hydrophilic polymers, proton-donating hydrophilic polymers, anionic compounds, cationic compounds, and zwitterionic compounds. In some embodiments, the coating 350 may include a wetting agent that may enhance wettability of the implantable medical device. In some embodiments, the wetting agent can include polyethers, including, for example, linear or branched polyethers (e.g., C₂-C₆-alkylene glycol). In some embodiments, the wetting agent can include polyethylene glycol (PEG) or similar non-ionic aprotic hydrophilic polymers. Suitable PEG can have an average molecular weight (Mn) from about 500 to about 20,000 Daltons (also, g/mol), more particularly, from about 4,000 to about 18,000, and even more particularly from about 6,000 to about 16,000 or from about 8,000 to about 14,000. In some embodiments, the wetting agent can be PEG having an average molecular weight of between about 10,000 and about 12,000. In some embodiments, the wetting agent can be PEG having an average molecular weight of about 500, 1,000, 2,000, 3,000, 4,000, 5,000, 6,000 7,000, 8,000, 9,000, 10,000, 11,000, 12,000, 13,000, 14,000, 15,000, 16,000, 17,000, 18,000, 19,000 or 20,000. In some embodiments, a coating comprising PEG having an average molecular weight from about 3,000 to about 5,000 is applied to the pulse generating portion of the implantable medical device, thus enhancing wettability of the medical device.

In some embodiments, the wetting agent can include PEG, polyethylene oxide (PEO), polyoxyethylene (POE), polypropylene glycol (PPG) and mixtures thereof. In some embodiments, the wetting agent can include block co-polymers comprising various combinations of at least one of PEG, PEO, POE and PPG and combinations thereof. For example, the wetting agent can be PEG-PPG or PEG-PPG-PEG, polydimethylsiloxane (PDMS) and/or polyvinylpyrrolidone (PVP). Suitable commercially available PEG-PPG-PEG co-polymers include Pluronic available from BASF of Spartanburg, S.C. The wetting agent can also include sodium carboxymethylcellulose, with or without PEG. In some embodiments, the wetting agent can comprise capped PEG. For example, suitable wetting agent includes Triton-X, a capped PEG (i.e., 4-(1,1,3,3-tetramethylbutyl)phenyl-polyethylene glycol, t-octylphenoxypolyethoxyethanol, polyethylene glycol tert-octylphenyl ether), available from Sigma-Aldrich of St. Louis, Mo.

In some embodiments, the wetting agent can include methoxy polyethyleneglycol methacrylate (MPEGMA) and its copolymers. In some cases, the wetting agent can include poly(methyl vinyl ether) and its copolymers. In some embodiments, the wetting agent can include polyoxazolines, poly-2-methyloxazoline and/or poly-2-ethyloxazoline, and their copolymers. In some embodiments, the wetting agent can include poly(N,N-dimethylacrylamide), and poly(N-vinylimidazole), and their copolymers and derivatives and combinations thereof.

In some embodiments, the wetting agent can include proton-donating hydrophilic polymers. For example, the wetting agent can include polyvinyl alcohol (PVA), 2-dodecenylsuccinic polyglyceride, poly(N-isopropylacrylamide) (PNIPAM), polyacrylamide (PAM) and their copolymers and combinations thereof. In some embodiments, the wetting agent can include polyacrylic acid, polyhexamethylene biguanide (PHMB), their copolymers and combinations thereof.

In some embodiments, the wetting agent can include anionic compounds. For example, the wetting agent can include poly-2-vinylpyrrolidone-alt-maleic anhydride, polyepoxysuccinic acid, poly(methyl vinyl ether-alt-maleic anhydride), poly(vinylphosphonic acid), poly(vinyl sulfate), poly(vinylsulfonic acid, sodium salt), polyanetholesulfonic acid, polystyrene sulfonate, alginic acid, pectic acid (polygalactouronic acid), sulfonated polysaccharides (e.g., heparin), and combinations thereof.

In other cases, the wetting agent can include cationic compounds or zwitterionic compounds. For example, the wetting agent can include poly(diallyldimethylammonium chloride), polyallylamine, poly[trialkyl(vinylbenzyl)ammonium chloride, polyphosphonium salts, polymers with quaternary ammonium substitution (e.g. poly(choline methacrylate)), poly(sulfobetaine methacrylate), poly(carboxybetaine methacrylate), and combinations thereof. Copolymers and derivatives of the components described herein and combinations thereof may also be suitable wetting agents.

In some embodiments, a coating comprising a wetting agent as described above can be applied to, and enhance the wettability of, a plurality of different substrate surfaces. For example, the coating can be applied to a substrate comprising platinum, platinum-iridium, iridium, titanium or alloys, tantalum, and other suitable metals.

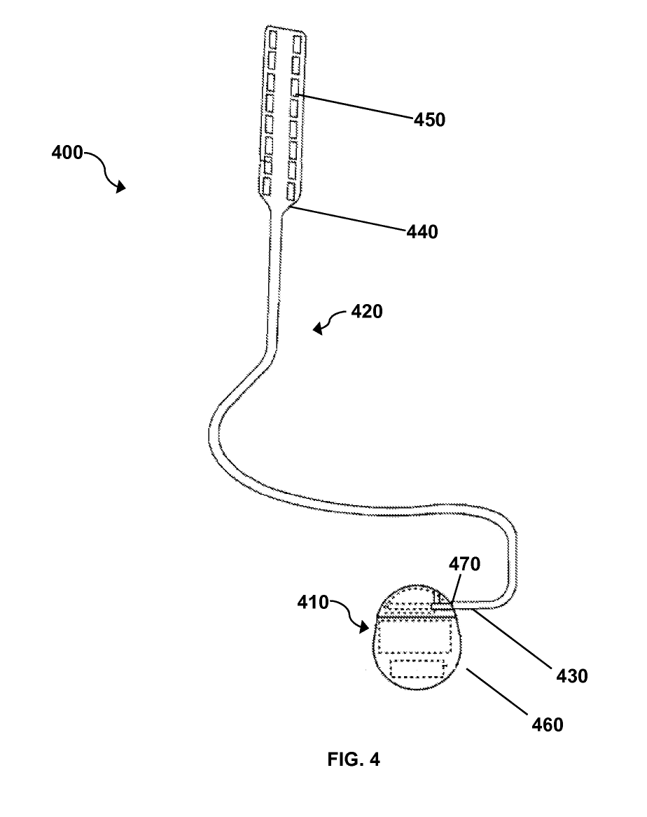

FIG. 4 is an exemplary illustration of an implantable medical device, according to one embodiment of the present invention. In some embodiments, the implantable medical device of the present invention can include an electrical stimulation system that modulates the electrical impulses in a subject. In some embodiments, the implantable medical device can be a neuromodulation device, as illustrated in FIG. 4. For example, the neuromodulation device 400 can include a pulse generating portion 410 and an active element 420. In the context of neuromodulation, the active element 420 can include an array of feedthough pins (i.e., feedthroughs) or an electrode array, shown in FIG. 4. The pulse generating portion 410 can be electrically coupled to the active element 420, which conveys electrical signals between the target tissue (e.g., muscle, neural tissue) and the pulse generating portion 410. A proximal region 430 of the active element 420 can be coupled to the pulse generating portion 410, and a distal region 440 can be coupled to the target tissue. In some embodiments, the distal region 440 can be terminated in an electrode array 450. The neuromodulation devices as illustrated in FIG. 4 can be used in neurological therapy by stimulating nerves or muscles. For example, stimulating nerve fibers proximal to the pudendal nerves of the pelvic floor can treat urinary urge incontinence, and stimulating the cavernous nerve(s) can be used to treat erectile and other sexual dysfunctions. Additionally, nerve stimulation can help reduce pressure sores and venous stasis, as well as stimulate the spinal cord for relieving intractable chronic pain.

In some embodiments, the neuromodulation device 400 can include a housing 460 which encloses pulse generating portion. In some embodiments, the housing 460 is a body that encases the electrical components (e.g., microprocessor chip, pulse output circuits, sense amplifiers, telemetry coils, etc.) necessary for sending a signal to the active element as well as a power source (e.g., a battery). In some embodiments, the walls of housing 460 may have substantially uniform thickness. The pulse generating portion 410 may be implanted subcutaneously at an implantation location near the target tissue. The housing 460 can be secured to the tissues of a subject during implantation to prevent migration of the pulse generating portion 410. The housing 460 can also encase a header portion 470, which generally provides an interface for connecting the active element 420 (e.g., electrode array or feedthrough) to the pulse generating portion 410 of the medical device.

In some aspects, the neuromodulation device 400 can further include a coating comprising a wetting agent, as previously described. The coating can be applied to the housing 460 of a pulse generating portion 410, to an active element 420, or both. The coating can be applied for a variety of purposes, including, but not limited to enhancing wettability, reducing electrical impedance, reducing bacterial adhesion, or reducing immune cell adhesion. In some cases, the coating comprises a wetting agent that can, for example, enhance wettability and reduce the variability in electrical impedance after implantation of the neuromodulation device 400. In other cases, the coating comprises a wetting agent that can, for example, reduce the ability of harmful bacteria to adhere to the medical device after implantation, thus exhibiting anti-microbial properties.

In some embodiments, lowering impedance of an implantable medical device system can shorten time required to reach a chronic steady state. For example, reducing the impedance of a medical device system can reduce the time required to reach a chronic steady state by days, weeks, or months. In some embodiments, an implantable medical device having enhanced wettability through the application of a coating which includes a wetting agent can reduce impedance or impedance variability, and lead to a reduction in the amount of time required to reach a chronic steady state.

In some embodiments, the coating can include one or more additional components such as, but not limited to, emulsifiers, surfactants and other suitable components and combinations thereof. In some embodiments, the coating can include an emulsifier or have emulsifying properties. Emulsifiers may stabilize an emulsion liquid or solution by increasing the kinetic stability of the emulsion. Emulsifiers may reduce surface tension and increase the viscosity of a liquid medium, which can help create and maintain homogeneity in immiscible liquids.

Additionally or alternatively, the coating can include a surface active agent (i.e., a surfactant) or have surfactant-like properties. Surfactants are generally considered to be a class of emulsifiers that help to lower the surface tension between two liquids or between a solid and a liquid. Surfactants may be classified according to their polar head group. A non-ionic surfactant has no charged groups in its head portion. The head of an ionic surfactant carries a net charge. If the charge of its head portion is negative, the surfactant is referred to as anionic; if the charge is positive, it is referred to as cationic. A surfactant having a head portion with two oppositely charged groups, is referred to as zwitterionic. Surfactants may include a polyether chain terminating in a highly polar anionic group. The polyether groups often include ethoxylated (polyethylene oxide-like) sequences inserted to increase the hydrophilic character of a surfactant. Polypropylene oxides conversely, may be inserted to increase the lipophilic character of a surfactant.

In some embodiments, the coating can include components in addition to or combination with the wetting agent.

Examples of other suitable components include but are not limited to small molecule drugs (e.g., growth factor inhibitors, anti-microbial agents), excipients, adjuvants, dyes, pharmaceutically effective carriers, chemical solvents, buffers, nanoparticles, and combinations thereof.

In some embodiments, the present invention provides a method of manufacturing an implantable medical device having a coating including a wetting agent. In some cases, the method can include applying a coating containing a wetting agent to at least one of the external surface of a pulse generating portion and an active element (e.g., lead or feedthrough). Additionally, the method can further include drying the coating applied to the at least one of the external surface of a pulse generating portion and an active element coupled to the pulse generating portion.

In some embodiments, the coating may be applied as a solution or mixture including the wetting agent and optionally one or more additional components. In some embodiments, the wetting agent is water-soluble or in a water-soluble form, such that it will readily dissolve in an aqueous solution without the need for additional mechanical or chemical assistance. In some embodiments, the solution or mixture can include a wetting agent in addition to various salts and/or buffering agents. For example, in some embodiments, one or more solvents can be used in order to form a coating solution for deposition on the surface of a portion of a medical device. Suitable solvents can include polar and non-polar solvents. For example, suitable solvents can include water, alcohols (such as, methanol, butanol, propanol, and isopropanol (isopropyl alcohol)), alkanes (such as, halogenated or unhalogenated alkanes such as chloroform, hexane, and cyclohexane), amides (such as, dimethylformamide), ethers (such as, THF and dioxolane), ketones (such as, acetone, methylethylketone), aromatic compounds (such as, toluene and xylene), nitriles (such as, acetonitrile) and esters (such as, ethyl acetate) and combinations thereof.

In some embodiments, the components of the coating solution can be dissolved in an aqueous solution before being applied to or disposed over the surface of a portion of a medical device. The coating solution may have a suitable viscosity such that it can be applied to a surface and dried thereon. Methods or techniques to apply the coating solution include, but are not limited to, dip-coating, spray-coating (such as gas-atomization and ultrasonic atomization), fogging, brush coating, press coating, blade coating, and other similar techniques. The coating solution may be applied to various substrates that are used for constructing implantable medical devices, including, but not limited to, metals, polymers, ceramics, and natural materials. Metals can include, but are not limited to, cobalt, chromium, nickel, titanium, tantalum, iridium, tungsten and alloys such as stainless steel, nitinol or cobalt chromium. Suitable metals can also include the noble metals such as gold, silver, copper, platinum, and alloys including the same.

In some embodiments, the coating solution can comprise PEG as the wetting agent, or derivatives and copolymers of PEG, as described herein. In some cases, the wetting agent can have an average molecular weight (Mn) in the range from about 500 to about 20,000 Daltons (also, g/mol). In some embodiments, the coating solution can be formulated such that, when applied and dried on the surface of an implantable medical device, PEG is present in a total amount of 1.0 mg or less. In some embodiments, application of the coating solution including the wetting agent can include spraying, followed by a period of time where the coating solution is allowed to dry. Drying may be determined visually (e.g., no visible moisture present), and the total amount of time in which a coating is allowed to dry varies with the different components that make up the coating. For example, the coating can be allowed to dry for about less than 1 hour. In some embodiments, the coating can be allowed to dry for about between 1 hour and about 3 hours. In other embodiments, the coating can be allowed to dry between about 3 hours and about 5 hours. In other embodiments, the coating can be allowed to dry for about 5 hours or more, and up to, for example, 24 hours. In some embodiments, drying the coating can be augmented or hastened through the use of drying facilitators, such as but not limited to fume hoods, desiccants and dehumidifiers.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

### EXAMPLES

The present invention is more particularly described in the following examples that are intended as illustrations only, since numerous modifications and variations within the scope of the present invention will be apparent to those of skill in the art.

### Example 1

### Electrical Impedance

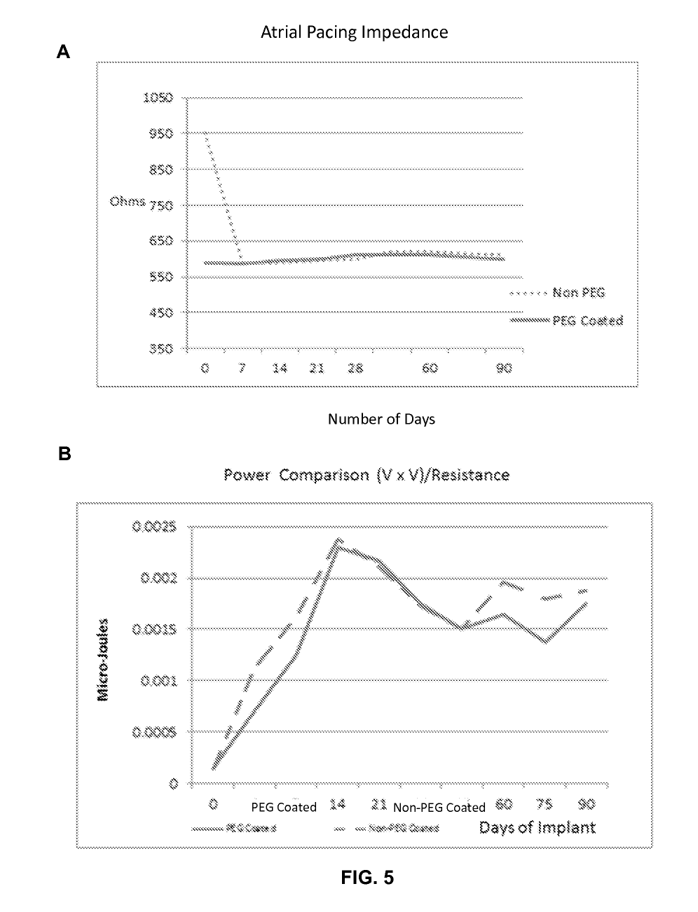

FIG. 5 part A is a graphical representation illustrating the variability in impedance between an initial phase (e.g., acute phase) after implantation of a medical device, and subsequent phases (e.g., chronic phases), when the electrical system stabilizes and reaches a chronic steady state.

Identical pacemaker pulse generators and atrial pacing leads were implanted into subjects. A coating containing polyethylene glycol (PEG) having an average molecular weight of 3350 as a wetting agent was applied to the atrial pacing leads of one of the pacing leads before implantation. Atrial pacing impedance was measured (Ohms, x-axis) over a period of time (days, y-axis).

As shown, atrial pacing impedance was reduced in the acute phase by at least about 5% in the leads/PGs having the PEG coating compared to the leads/PGs without the PEG coating. Additionally, the variability in atrial pacing impedance was reduced from the acute phase through to the chronic phase by at least about 5% in the leads/PGs having the PEG coating compared to the leads/PG without the PEG coating.

FIG. 5 part B is a graphical representation comparing the changes in power between an implantable medical device comprising a coating containing PEG having an average molecular weight of 3350 and an uncoated medical device. In this exemplary embodiment, the implantable medical device comprising the PEG coating exhibits reduced power due to reduced threshold voltage compared to the uncoated medical device during the acute phase as well as the chronic phase. The reduction in power required to operate the medical device can occur in part due to a reduction in electrical impedance or impedance variability caused by, for example, improved wetting of at least a portion of the medical device. In some embodiments, reducing the power required to operate an implantable medical device, as shown in FIG. 5 part B, can lead to better medical outcomes for a patient because less medical intervention may be needed to stabilize the implantable medical device after the acute phase post-implantation. Additionally, reducing the power required to operate a medical device can extend the operational life of the medical device and thus contribute to an improvement in the patient's quality of life.

### Example 2

### Bacterial Adhesion

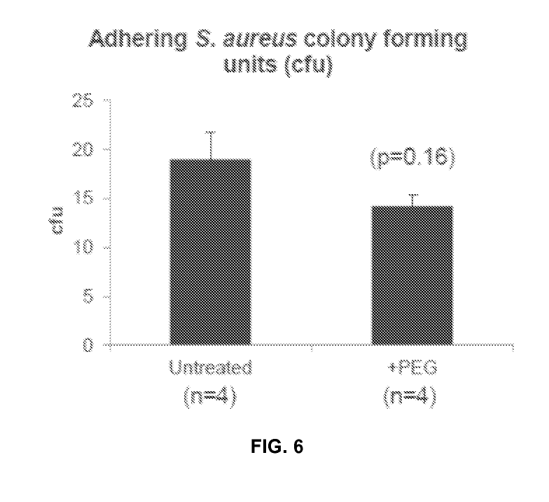

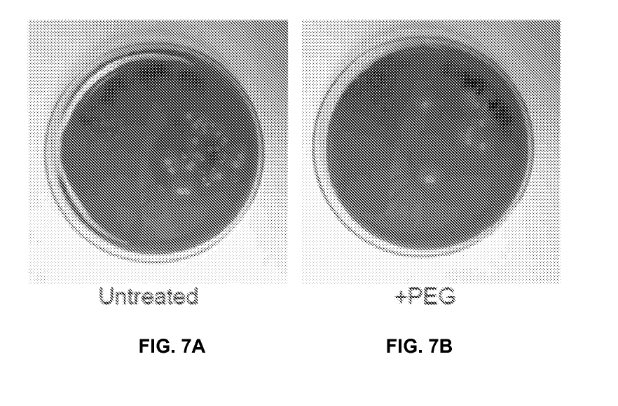

FIG. 6 is a graphical representation illustrating reduced bacterial adhesion to a portion of an implantable medical device having enhanced wettability through the application of a coating comprising a wetting agent. A coating comprising the wetting agent PEG having an average molecular weight of 3350 was applied to five identical pulse generating portions of cardiac pacemakers, while five other identical pulse generating portions were left uncoated (i.e., controls). Each pulse generating portion was allowed to dry in a fume hood after receiving the coating. Less than about 1 mg total of PEG was applied to each pulse generating portion as determined by weighing the pulse generating portions before and after the coating was applied. Each pulse generating portion was exposed to a 3 ml suspension of log phase bacteria (i.e., S. aureus) in the well of a 6-well plate for 2 hours while rocking at room temperature. Following exposure, loosely adhered bacteria were removed by rigorous washing by vortex in CMF-PBS. Each pulse generating portion was then stamped on a TSA plate and bacterial growth was assessed 24 hours after stamping (see FIGS. 7A and 7B).

As shown in FIG. 6, pulse generating portions coated with PEG produced a lower number of colony forming units (cfu) compared to uncoated controls. This indicates that coating a portion of an implantable medical device with a coating that includes a wetting agent (e.g., PEG) can reduce the ability of harmful bacteria to adhere to that portion of the medical device. This anti-microbial characteristic may reduce the likelihood of injections.

FIGS. 7A and 7B are exemplary images of TSA plates stamped with a portion of an implantable medical device that was either untreated (FIG. 7A) or treated (FIG. 7B) with a coating that included the wetting agent PEG. As noted above, the treated and untreated portions of the medical devices were stamped on TSA plates (after rigorous washing) and the numbers of cfu were quantified.

In some embodiments, the wetting agent may be present on at least a portion of the medical device in an amount of about 1.0 mg or less. For example, the wetting agent may be present on the housing in an amount of about 1.0 mg or less. In some aspects, the wetting agent may be present on the medical device in a total amount of about 1.0 mg or less. For example, the combined or total about of the wetting agent present on the exterior surface of the medical device (including the exterior surface of the housing and the lead) may be about 1.0 mg or less. In some embodiments, the wetting agent may be present on the exterior surface of the device in a total amount from about 0.1 mg to about 0.5 mg. In other embodiments, the wetting agent may be present on the exterior surface of the device in a total amount from about 0.5 mg to about 1.0 mg.

In some embodiments, the wetting agent is selected such that the coating forms a cross-linkable hydrophilic polymer on the surface of the medical device. For example, suitable wetting agents may include PEG in a cross-linkable form. In some embodiments, the wetting agent can be PEG having reactive side chains that enable the formation of cross-linked macromers, such that the coating forms a cross-linkable hydrophilic polymer on a surface of the medical device. In other embodiments, suitable wetting agents can be chosen such that the coating does not form a cross-linked hydrophilic polymer on the surface of a medical device, despite being cross-linkable (e.g., PEG lacking reactive side chains). In some embodiments, the wetting agent can form a cross-linkable hydrophilic polymer that will form hydrogels capable of swelling in water or biological fluids or tissues on the surface of a surface of the medical device. In other embodiments, the wetting agent can form a cross-linkable hydrophilic polymer that will not form hydrogels, and will not swell in the presence of water or biological fluids or tissues.

We claim:

1. An implantable medical device comprising:
a pulse generating portion enclosed within a housing;
an active element electrically coupled to the pulse generating portion; and
a coating comprising a wetting agent, the coating disposed over at least a portion of at least one of the housing of the pulse generating portion and the active element, wherein the wetting agent comprises polyethylene glycol (PEG) having an average molecular weight from about 500 to about 20,000 Daltons, and wherein the PEG is present in an amount of about 1 mg or less.

2. The implantable medical device of claim 1, wherein the PEG comprises a cross-linkable hydrophilic polymer.

3. The implantable medical device of claim 1, wherein the electrical impedance is reduced by at least about 5% compared to the medical device without the coating.

4. The implantable medical device of claim 1, wherein the variability in electrical impedance between the acute phase and the chronic phase is reduced compared to the medical device without the coating.

5. An implantable medical device comprising:
a pulse generating portion enclosed within a housing;
an active element, wherein the pulse generating portion is electrically coupled to the active element; and
a coating comprising a wetting agent, the coating disposed over at least a portion of the medical device,
wherein electrical impedance of the implantable medical device is reduced compared to the medical device without the coating.

6. The implantable medical device of claim 5, wherein the coating is disposed over at least a portion of at least one of the housing of the pulse generating portion and the active element.

7. The implantable medical device of claim 5, wherein the electrical impedance is reduced by at least about 5% compared to the medical device without the coating.

8. The implantable medical device of claim 5, wherein the variability in electrical impedance between the acute phase and the chronic phase is reduced compared to the medical device without the coating.

9. The implantable medical device of claim 5, wherein the wetting agent comprises a member selected from the group consisting of polyethylene glycol (PEG), PEG copolymers and combinations thereof.

10. The implantable medical device of claim 9, wherein the wetting agent is PEG having an average molecular weight from about 500 and about 20,000 Daltons.

11. The implantable medical device of claim 10, wherein the PEG applied is present in a total amount of about 1 mg or less.

12. The implantable medical device of claim 5, wherein the wetting agent is a cross-linkable hydrophilic polymer.

13. The implantable medical device of claim 5, wherein the coating reduces interfacial surface tension between an aqueous fluid and the external surface of at least a portion of the implantable medical device.

14. The implantable medical device of claim 5, wherein the coating reduces bacterial adhesion compared to a medical device not having the coating.

15. The implantable medical device of claim 5, further comprising a chamber covering the pulse generation portion and the housing of the pulse generating portion, wherein the coating is applied to the external surface of the chamber instead of the housing.

16. The implantable medical device of claim 5, wherein the active element is a lead.

17. A method of manufacturing an implantable medical device, the method comprising:
applying a coating solution comprising a wetting agent to an external surface of the medical device; and
drying the applied coating solution;
wherein electrical impedance of the medical device is reduced compared to the medical device without the coating.

18. The method of claim 17, wherein applying the coating solution comprises brushing, dipping, spraying, or combinations thereof.

19. The method of claim 17, wherein the electrical impedance is reduced by at least about 5% compared to the medical device without the coating.

20. The method of claim 17, wherein the wetting agent is PEG having an average molecular weight from about 500 to about 20,000 Daltons.
